(19) United States
(12) Patent Application Publication
(10) Pub. No.: US 2024/0161875 A1
(43) Pub. Date: May 16, 2024

(54) MACHINE LEARNING SYSTEM FOR PREDICTING BIOMARKERS

(71) Applicant: CVS Pharmacy, Inc., Woonsocket, RI (US)

(72) Inventors: Eric Hwai-Yu Yang, Warminster, PA (US); Samta Shukla, Lexington, MA (US); Jian Su, Hoboken, NJ (US); Yoon Hyeok Yang, Cambridge, MA (US)

(21) Appl. No.: 18/388,414

(22) Filed: Nov. 9, 2023

Related U.S. Application Data
(60) Provisional application No. 63/424,739, filed on Nov. 11, 2022.

(51) Int. Cl. G16B 40/20; G06N 3/08; G16H 50/20

(52) U.S. Cl. CPC G16B 40/20; G06N 3/08; G16H 50/20

(57) ABSTRACT

A system, method, and apparatus are provided that include: providing a dataset to a machine learning model, where the dataset includes claims-based electronic data; receiving an output from the machine learning model in response to the machine learning model processing at least a portion of the dataset, where the output includes a predicted value of a biomarker; processing the portion of the dataset for identifying information associated with an individual in response to determining the predicted value of the biomarker satisfies one or more criteria; and transmitting, via a communication network to one or more communication devices, an electronic communication including information associated with the predicted value of the biomarker.

### CROSS REFERENCE TO RELATED APPLICATIONS

The present application claims the benefit of and priority to U.S. Provisional Application No. 63/424,739, filed on Nov. 11, 2022, entitled “MACHINE LEARNING SYSTEM FOR PREDICTING BIOMARKERS,” which application is incorporated herein by reference in its entirety.

### FIELD OF THE DISCLOSURE

The disclosure relates to systems and methods for predicting biomarkers of individuals, and more particularly, Artificial Intelligence (AI) based systems and methods for quantifying health statuses of the individuals based on the predicted biomarkers.

### BACKGROUND

Pharmaceutical manufacturers, healthcare providers, pharmaceutical distributors, and other entities may provide medical assessments of individuals with respect to biomarker data. Improved techniques for providing the medical assessments are desired.

### SUMMARY

A method described herein includes: providing a dataset to a machine learning model, wherein the dataset includes claims-based electronic data; receiving an output from the machine learning model in response to the machine learning model processing at least a portion of the dataset, wherein the output includes a predicted value of a biomarker; processing the portion of the dataset for identifying information associated with at least one individual in response to determining the predicted value of the biomarker satisfies one or more criteria; and transmitting, via a communication network to one or more communication devices, an electronic communication including information associated with the predicted value of the biomarker.

In some aspects, the claims-based electronic data is associated with a group of individuals; and the biomarker is associated with individuals in the group of individuals.

In some aspects, the dataset includes prescription-based electronic data.

In some aspects, the biomarker includes hemoglobin A1C.

In some aspects, the dataset does not include measured values of the biomarker.

In some aspects, the output includes: a predicted health status of the at least one individual corresponding to the predicted value of the biomarker.

In some examples, the predicted health status includes at least one of: a medical condition of the at least one individual; a predicted risk of the at least one individual with respect to developing the medical condition; and a predicted severity of the medical condition.

Some examples include providing a training dataset to the machine learning model, wherein the training dataset includes: claims-based electronic data of a set of reference individuals; prescription-based electronic data of the set of reference individuals; measured biometric data of the set of reference individuals, wherein the measured biometric data includes reference measured values of the biomarker; and diagnosed medical conditions associated with the set of reference individuals.

In some aspects, the input includes one or more candidate intervention actions. In some aspects, the output includes a predicted effect of each of the one or more candidate interventions, wherein the predicted effect includes: a second predicted value of the biomarker; and a temporal value associated with achieving the second predicted value.

In some aspects, the input includes one or more candidate treatment plans. In some aspects, the output includes a predicted effect of each of the one or more candidate treatment plans, wherein the predicted effect includes: a second predicted value of the biomarker; and a temporal value associated with achieving the second predicted value.

In some aspects, the one or more candidate treatment plans include at least one of: a medical procedure; an action of the at least one individual; and a drug regimen.

In some aspects, the output includes respective rankings corresponding to: closing a care gap associated with the at least one individual; implementing a candidate intervention action; and implementing a candidate treatment plan.

Some examples include: providing a target medical condition to the machine learning model; receiving a second output from the machine learning model in response to providing the target medical condition, wherein the second output includes: one or more biomarkers indicative of the target medical condition; and a predicted value of the one or more biomarkers; processing the portion of the dataset for the identifying information associated with the at least one individual in response to determining the predicted value of the one or more biomarkers satisfies one or more second criteria; and transmitting, via the communication network to the one or more communication devices, a second electronic communication including information associated with the predicted value of the one or more biomarkers.

In some aspects, the second output includes at least one of: one or more claims-based identifiers indicative of the target medical condition; and one or more prescription-based identifiers indicative of the target medical condition.

In some aspects, the claims-based electronic data includes Current Procedural Terminology (CPT) codes and National Drug Code (NDC) numbers.

In some aspects, the output includes a range of values of the biomarker.

In some aspects, the output includes a predicted value of one or more additional biomarkers associated with the at least one individual.

In some aspects, the electronic communication includes at least a portion of the output from the machine learning model.

In some aspects, the one or more communication devices include a communication device of the at least one individual, a communication device of a care provider of the at least one individual, or both.

All examples and features mentioned above can be combined in any technically possible way.

### BRIEF DESCRIPTION OF THE DRAWINGS

The present disclosure is described in conjunction with the appended figures, which are not necessarily drawn to scale:

FIG. 1 illustrates an example of a system in accordance with aspects of the present disclosure.

FIG. 2 illustrates a block diagram that supports aspects of the present disclosure.

FIG. 3 illustrates an example of a neural network architecture in accordance with aspects of the present disclosure.

FIGS. 4A through 4C illustrate example aspects of a neural network architecture in accordance with aspects of the present disclosure.

FIG. 5 illustrates an example process flow in accordance with aspects of the present disclosure.

### DETAILED DESCRIPTION

Before any examples of the disclosure are explained in detail, it is to be understood that the disclosure is not limited in its application to the details of construction and the arrangement of components set forth in the following description or illustrated in the following drawings. The disclosure is capable of other configurations and of being practiced or of being carried out in various ways. Also, it is to be understood that the phraseology and terminology used herein is for the purpose of description and should not be regarded as limiting.

While various examples will be described in connection with predicting a value of a biomarker (e.g., hemoglobin A1C) of an individual and further predicting a corresponding health status (e.g., risk or severity of diabetes) of the individual based on the value of the biomarker, it should be appreciated that the disclosure is not so limited. For instance, it is contemplated that examples of the present disclosure can be applied to predicting values of any biomarkers (e.g., cholesterol level, blood pressure, heart rate, bone mineral density, etc.) and using the biomarker values to gauge or evaluate any medical condition or health status that is quantifiable from the biomarker values. Indeed, examples of the present disclosure can be applied to or used in connection with biomarkers having molecular, histologic, radiographic, and/or physiological characteristics.

Example medical conditions include diabetes, cardiac conditions, heightened cholesterol, heightened blood pressure, hypertension, post-operative conditions, pre-operative conditions, cancer and other chronic conditions, infertility, chronic pain, broken bones, torn ligaments, torn muscles, and the like, and are not limited thereto. In other words, the framework described herein for predicting values of biomarkers using claims-based electronic data and/or prescription-based electronic data can be leveraged to support care management opportunities in association with any type or number of different medical conditions.

The terms “member,” “patient,” “individual,” and “subject” may be used interchangeably herein. The terms “care gap” or “gap-in-care” may be used interchangeably herein. The terms “lab value” and “measured biomarker value” may be used interchangeably herein.

In some medical environments, diseases or assessments of individual health may be given with respect to biomarkers, such as estimated average glucose (also referred to herein as A1C), cholesterol level, blood pressure, and the like. However, in some cases, the amount of measured biomarker data available for assessing the health of a member may be limited or unavailable. Accordingly, for example, the data available to a medical provider may be incomplete. For example, while a medical provider or other healthcare entity may have complete claims data, prescription data, and medical procedure data for a member, cases in which the amount of measured biomarker data is relatively limited (e.g., a relatively low amount of measured biomarker data is available) may prevent accurate assessments of the health of the member. In some cases, such limited amounts of measured biomarker data may prevent assessments of the severity of the disease burden of the member.

In many cases, biomarker data associated with the member is unavailable. For instance, in many Real World Data (RWD) datasets, biomarker data for the majority of individuals will be missing. In some cases, the absence of the biomarker data may negatively impact the ability of a medical provider or other healthcare entity to accurately provide assessments of disease or disease severity. Aspects of the present disclosure described herein provide techniques for predicting biomarker values from claims or other types of procedure data, which may greatly increase the value of such RWD datasets and open up numerous avenues by which patient health and/or the results of medical interventions can be evaluated upon a macro level.

Aspects of the present disclosure support deep learning models that provide an avenue by which a medical provider and/or insurance provider can assess member health and treatment effectiveness. For example, aspects of the present disclosure support at least the following example goals of medical providers and/or insurance providers: to properly assess future medical costs of new and existing members, and, to identify and prioritize treatments that would most benefit the health of a member.

According to example aspects of the present disclosure, techniques described herein support accurate prediction of biomarker values (e.g., A1C values, cholesterol levels, blood pressure values, etc.) of a member from medical claims associated with the member. For example, the techniques may include predicting the biomarker values from Current Procedural Terminology (CPT) codes and National Drug Code (NDC) codes.

A system described herein may support an imputation method for uncaptured biomarker data and, in some cases, may provide more than a causal model. For example, the system may predict biomarker values (e.g., A1C values, cholesterol levels, blood pressure values, etc.) and, using the predicted biomarker values, identify individuals who are at risk of a health condition for treatment intervention. In an example, the system may predict A1C values and, using the predicted A1C values, identify individuals who are at risk of uncontrolled diabetes for treatment intervention.

In some aspects, the system may support identifying CPT codes (e.g., medical services and procedures) and NDC codes (e.g., prescriptions) that are predictive of an individual with a health condition that may arise in the future. For example, the system may support identifying CPT codes and NDC codes that are indicative of an individual with uncontrolled diabetes. Accordingly, for example, the system supports clinical markers that can be used in a computationally efficient model for predicting the health of individuals.

According to example aspects of the present disclosure, the techniques described herein support estimating values of biomarkers (e.g., A1C) for members for which no values of the biomarker have been reported, using claims data and prescription data. The techniques include a machine learning method to quantify health statuses of members (e.g., identifying members at risk of having diabetes or understanding the severity of their diabetes) without having access to lab values for the members. In some example cases, lab values for a member may be unavailable because the member has failed to get tested.

In some other example cases, the member may have been tested, but the lab values are only available by the facility that performed the test. For example, a lab that is partnered with a healthcare provider (or insurance provider) may share an outcome of a test (e.g., pass, fail or other outcome) for an individual but refrain from sharing measured biomarker values associated with the test. Accordingly, for example, aspects of the present disclosure provide advantages for cases in which lab values and measured biomarker data are unavailable or cannot be accessed.

Aspects of the techniques described herein support gauging, using the predicted biomarker values, the severity of health conditions for members. In some cases, the techniques described herein support using the predicted biomarker values to identify care gaps for the members in association with the health conditions. Accordingly, for example, the techniques described herein may assist in identifying interventions for members in association with managing a health condition. Example interventions include communications (e.g., email communications, SMS messaging, personal visits, etc.) for encouraging a member to get tested or otherwise manage their diabetes (or other health condition).

In some other aspects, the techniques described herein may support identifying actions (e.g., medical procedures, exercise regimens, etc.) and medications that impact biomarker values (e.g., reduce A1C values). For example, a system may recommend identified actions and/or medications as interventions to members in association with managing health conditions of the members.

In some example implementations, the techniques described herein may be extrapolated to other types of biomarkers and health conditions. For example, the present disclosure may support machine learning techniques capable of predicting lab values that might be indicative of a given disease/health condition. Accordingly, for example, for a target health condition to be monitored, the system may identify a biomarker and corresponding lab values of the biomarker that may indicate the presence or absence of the target health condition.

The techniques described herein provide advantages over other systems which may rely on reported lab values. For example, some other systems may determine a health status of a member based on lab reported values of a biomarker, but are unable to predict values of the biomarker for cases in which lab reported values are unavailable or partially available.

The techniques described herein may provide improved risk assessments (e.g., increased accuracy of a diagnosis, etc.) corresponding to members for cases in which lab values are unavailable. In some other cases, the techniques described herein may support reduced overhead in association with managing member care (e.g., reduced testing costs, reduced monitoring, improved convenience to a member, etc.), as the capability to predict values of biomarkers based on claims data and/or prescription data may support a reduced number of lab tests. In some aspects, the techniques described herein may support prioritized medical treatment for members, as the capability to predict values of biomarkers based on claims data and/or prescription data may support identification of members with severe health conditions. In some other aspects, the techniques described herein may support identifying effective lower cost alternatives to prescribed treatment.

Example aspects of the present disclosure are described with reference to the following figures.

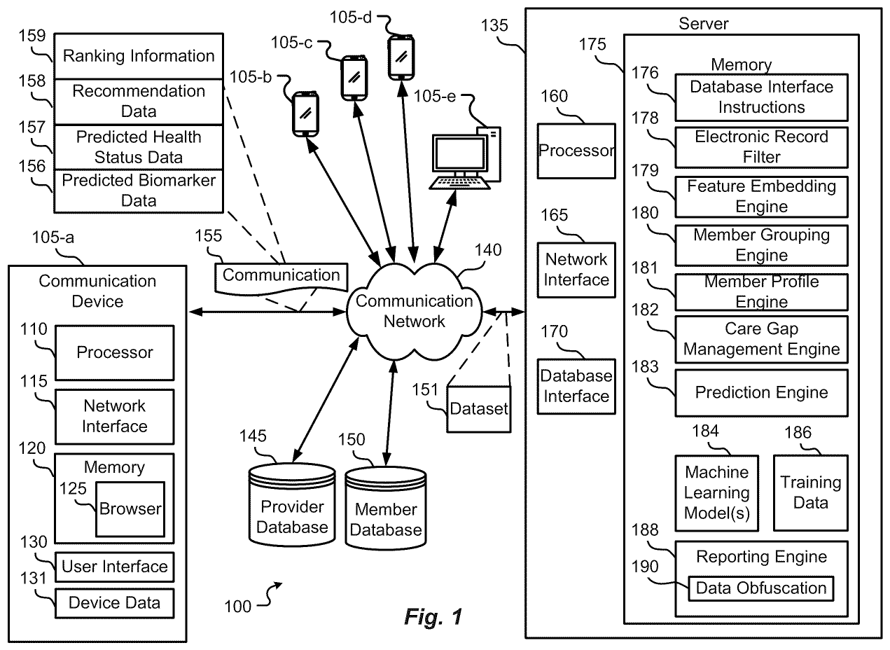

FIG. 1 illustrates an example of a system 100 in accordance with aspects of the present disclosure. The system 100, in some examples, may include one or more computing devices operating in cooperation with one another to predict biomarker values of a member and quantify a health status of the member based on the biomarker values. The system 100 may be, for example, a healthcare management system.

The components of the system 100 may be utilized to facilitate one, some, or all of the methods described herein or portions thereof without departing from the scope of the present disclosure. Furthermore, the servers described herein may include example components or instruction sets, and aspects of the present disclosure are not limited thereto. In an example, a server may be provided with all of the instruction sets and data depicted and described in the server of FIG. 1. Alternatively, or additionally, different servers or multiple servers may be provided with different instruction sets than those depicted in FIG. 1.

The system 100 may include communication devices 105 (e.g., communication device 105-a through communication device 105-e), a server 135, a communication network 140, a provider database 145, and a member database 150. The communication network 140 may facilitate machine-to-machine communications between any of the communication device 105 (or multiple communication devices 105), the server 135, or one or more databases (e.g., a provider database 145, a member database 150). The communication network 140 may include any type of known communication medium or collection of communication media and may use any type of protocols to transport messages between endpoints. The communication network 140 may include wired communications technologies, wireless communications technologies, or any combination thereof.

The Internet is an example of the communication network 140 that constitutes an Internet Protocol (IP) network consisting of multiple computers, computing networks, and other communication devices located in multiple locations, and components in the communication network 140 (e.g., computers, computing networks, communication devices) may be connected through one or more telephone systems and other means. Other examples of the communication network 140 may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communication network 140 may include any combination of networks or network types. In some aspects, the communication network 140 may include any combination of communication mediums such as coaxial cable, copper cable/wire, fiber-optic cable, or antennas for communicating data (e.g., transmitting/receiving data).

A communication device 105 (e.g., communication device 105-a) may include a processor 110, a network interface 115, a computer memory 120, a user interface 130, and device data 131. In some examples, components of the communication device 105 (e.g., processor 110, network interface 115, computer memory 120, user interface 130) may communicate over a system bus (e.g., control busses, address busses, data busses) included in the communication device 105. In some cases, the communication device 105 may be referred to as a computing resource. The communication device 105 may establish one or more connections with the communication network 140 via the network interface 115. In some cases, the communication device 105 may transmit or receive packets to one or more other devices (e.g., another communication device 105, the server 135, the provider database 145, the provider database 150) via the communication network 140.

Non-limiting examples of the communication device 105 may include, for example, personal computing devices or mobile computing devices (e.g., laptop computers, mobile phones, smart phones, smart devices, wearable devices, tablets, etc.). In some examples, the communication device 105 may be operable by or carried by a human user. In some aspects, the communication device 105 may perform one or more operations autonomously or in combination with an input by the user.

The communication device 105 may support one or more operations or procedures associated with predicting values of biomarkers of members and quantifying health statuses of the members based on the biomarker values, as described herein. For example, the communication device 105 may support communications between multiple entities such as a healthcare provider, a medical insurance provider, a pharmaceutical manufacturer, a pharmaceutical distributor, a member, or combinations thereof. In some cases, the system 100 may include any number of communication devices 105, and each of the communication devices 105 may be associated with a respective entity.

The communication device 105 may render or output any combination of notifications, messages, reports, menus, etc. based on data communications transmitted or received by the communication device 105 over the communication network 140. For example, the communication device 105 may receive one or more electronic communications 155 (e.g., from the server 135) via the communication network 140. Additionally, or alternatively, the system 100 may support communications of any electronic communications 155 between any device of the system 100, and the electronic communications 155 may include any combination of transmitted or received data as described herein.

In some aspects, the communication device 105 may render a presentation (e.g., visually, audibly, using haptic feedback, etc.) of the electronic communication 155 via the user interface 130. The user interface 130 may include, for example, a display, an audio output device (e.g., a speaker, a headphone connector), or any combination thereof. In some aspects, the communication device 105 may render a presentation using one or more applications (e.g., a browser application 125) stored on the memory 120. In an example, the browser application 125 may be configured to receive the electronic communication 155 in an electronic format (e.g., in an electronic communication via the communication network 140) and present content of the electronic communication 155 via the user interface 130.

In some aspects, the server 135 may communicate the electronic communication 155 to a communication device 105 (e.g., communication device 105-a) of a member, a communication device 105 (e.g., communication device 105-b) of a healthcare provider, a communication device 105 (e.g., communication device 105-c) of an insurance provider, a communication device 105 (e.g., communication device 105-d) of a pharmacist or pharmacy, a communication device 105 (e.g., communication device 105-e) of team outreach personnel, or the like. Additionally, or alternatively, the server 135 may communicate a physical representation (e.g., a letter) of the electronic communication 155 to the member, a healthcare provider, an insurance provider, a pharmacist, team outreach personnel, or the like via a direct mail provider (e.g., postal service).

In some aspects, the electronic communication 155 may include predicted biomarker data 156, predicted health status data 157, recommendation data 158, and ranking information 159.

The predicted biomarker data 156 may include a predicted value of a biomarker associated with a member. For example, the biomarker may be hemoglobin A1C. Additionally, or alternatively, the biomarker may include a cholesterol level, blood pressure, or the like, and is not limited thereto.

The predicted health status data 157 may include a predicted health status of the member corresponding to the predicted value of the biomarker. In an example, the predicted health status data 157 may include a medical condition of a member, a predicted risk of the member with respect to developing the medical condition, and a predicted severity of the medical condition.

The recommendation data 158 may include an indication of care gaps associated with a member and a predicted health status. The recommendation data 158 may include candidate intervention actions (e.g., candidate treatment interventions associated with closing a care gap and addressing a predicted health status). The candidate intervention actions may include outreach actions for encouraging the member to follow a treatment plan.

The recommendation data 158 may candidate treatment plans associated with addressing closing a care gap and addressing a predicted health status. Examples of the candidate treatment plans include, but are not limited to, a medical procedure, an action of the member (e.g., diet, exercise, etc.), and a drug regimen.

In some cases, the recommendation data 158 may include a predicted effect of closing a care gap, a predicted effect of a candidate intervention action, and/or a predicted effect of a candidate treatment plan. Examples of the predicted effect may include a predicted biomarker value (also referred to herein as a second predicted biomarker value) associated with closing a care gap, implementing a candidate intervention action, and/or a implementing a candidate treatment plan. In some examples, the predicted effect may include a temporal value associated with achieving the second predicted biomarker value (e.g., a predicted temporal duration until achieving the second predicted biomarker value). In some cases, the ranking information 159 may include respective rankings of closing a care gap(s) associated with a member, implementing a candidate intervention action(s), and implementing a candidate treatment plan(s).

Examples of the predicted biomarker data 156, predicted health status data 157, and recommendation data 158 are later described herein.

The provider database 145 and the member database 150 may include member electronic records (also referred to herein as a data records) stored therein. In some aspects, the electronic records may be accessible to a communication device 105 (e.g., operated by healthcare provider personnel, insurance provider personnel, a member, a pharmacist, etc.) and/or the server 135. In some aspects, a communication device 105 and/or the server 135 may receive and/or access the electronic records from the provider database 145 and the member database 150 (e.g., based on a set of permissions). In an example, the communication device 105 and/or server 135 may access a dataset 151 (e.g., associated with a member or members) from the provider database 145 and/or the member database 150.

In some other aspects, the electronic records may include device data 131 obtained from a communication device 105 (e.g., communication device 105-a) associated with the member. For example, the device data 131 may include gyroscopic data, accelerometer data, beacon data, glucose readings, heart rate data, blood pressure data, blood oxygen data, temperature data, kinetics data, location data, motion data, a device identifier, and/or temporal data (e.g., a timestamp) measurable, trackable, and/or providable by the communication device 105 (or a device connected to the communication device 105) associated with the member. In some aspects, the device data 131 may include information associated with a structured diet plan and/or exercise plan. For example, the device data 131 may include data logged in association with a diet logging application and/or exercise logging application executed at the communication device 105.

In some aspects, the electronic record may include an image of the member. For example, the electronic record may include imaging data based on which the server 135 (e.g., the care gap management engine 182) may track targeted biomarkers. For example, the server 135 may track X-ray records of a member over time (e.g., in associated with assisting reduced healing times for a member). In some cases, the electronic record may include other types of diagnostic images such as magnetic resonance imaging (MM) scans, computed tomography scans (CT), ultrasound images, or the like.

In some aspects, the dataset 151 may include electronic medical record (EMR) data. The dataset 151 may include data describing an insurance medical claim, pharmacy claim, and/or insurance claim made by the member and/or a medical provider. Accordingly, for example, the dataset 151 may come from providers or payers, and claims included in the claims-based electronic data may be of various types (e.g., medical, pharmacy, etc.).

In accordance with aspects of the present disclosure, the device data 131 may be provided continuously, semi-continuously, periodically, and/or based on a trigger condition by the communication device 105 (e.g., a smart watch, a wearable monitor, a self-reporting monitor such as a glucometer, a smartphone carried by a user, etc.) around monitored parameters such as heartbeat, blood pressure, etc. In some aspects, the device data 131 of a communication device 105 (e.g., communication device 105-a) may be referred to as “environmental data” associated with a user, which may be representative of aspects of environmental factors (e.g., lifestyle, socioeconomic factors, details about the environment, etc.) associated with a member.

In some aspects, the electronic records may include genetic data associated with a member. In some other aspects, the electronic record may include notes/documentation that is recorded at a communication device 105 in a universal and/or systematic format (e.g., subjective, objective, assessment, and plan (SOAP) notes/documentation) among medical providers, insurers, etc. In some examples, the electronic records may include non-claim adjudicated diagnoses input at a communication device 105 (e.g., diagnoses that have not been evaluated by an insurance provider with respect to payment of benefits).

In some other aspects, the electronic records may be inclusive of aspects of a member's health history and health outlook. The electronic records may include a number of fields for storing different types of information to describe the member's health history and health outlook. As an example, the electronic records may include personal health information (PHI) data. The PHI data may be stored encrypted and may include member identifier information such as, for example, name, address, member number, social security number, date of birth, etc. In some aspects, the electronic records may include treatment data such as, for example, member health history, member treatment history, lab test results (e.g., text-based, image-based, or both), pharmaceutical treatments and therapeutic treatments (e.g., indicated using predefined healthcare codes, treatment codes, or both), insurance claims history, healthcare provider information (e.g., doctors, therapists, etc. involved in providing healthcare services to the member), in-member information (e.g., whether treatment is associated with care), location information (e.g., associated with treatments or prescriptions provided to the member), family history (e.g., inclusive of medical data records associated with family members of the member, data links to the records, etc.), or any combination thereof. In some aspects, the electronic records may be stored or accessed according to one or more common field values (e.g., common parameters such as common healthcare provider, common location, common claims history, etc.). In some aspects, the system 100 may support member identifiers based on which a server 135 and/or a communication device 105 may access and/or identify key health data per member different from the PHI data.

In some aspects, the gap-in-care described herein may be defined by a difference between guideline behavior associated with what a member should be doing, as defined by clinical guidelines and expert clinical opinion (e.g., professional guidelines surrounding preventative screenings and close follow-up and monitoring with healthcare providers) and current health related behavior associated with what the member is actually doing, which may be defined by static or longitudinal observables in the medical history of the member and supporting data.

In some aspects, the provider database 145 may be accessible to a healthcare provider of a member (also referred to herein as a member), and in some cases, include member information associated with the healthcare provider that provided a treatment to the member. In some aspects, the provider database 145 may be accessible to an insurance provider associated with the member. The member database 150 may correspond to any type of known database, and the fields of the electronic records may be formatted according to the type of database used to implement the member database 150. Non-limiting examples of the types of database architectures that may be used for the member database 150 include a relational database, a centralized database, a distributed database, an operational database, a hierarchical database, a network database, an object-oriented database, a graph database, a NoSQL (non-relational) database, etc. In some cases, the member database 150 may include an entire healthcare history or journey of a member, whereas the provider database 145 may provide a snapshot of a member's healthcare history with respect to a healthcare provider. In some examples, the electronic records stored in the member database 150 may correspond to a collection or aggregation of electronic records from any combination of provider databases 145 and entities involved in the member's healthcare delivery (e.g., a pharmaceutical distributor, a pharmaceutical manufacturer, etc.).

The provider database 145 and/or the member database 150 may include chronic disease indicators recorded for each member using a database format associated with the provider database 145 and/or the member database 150. In some aspects, the provider database 145 and/or the member database 150 may support diagnosis and procedure codes classified according to the International Classification of Diseases 10th revision (ICD-10) and Current Procedure Terminology 4th revision (CPT-4) codes. In some aspects, the provider database 145 and/or the member database 150 may support the use of Generic Product Identifier (GPI) and National Drug Code (NDC) Directory information for common diabetes medications. The provider database 145 and/or member database 150 may include demographic information, including age, gender, race, and geography, identified using claims data. The provider database 145 and/or member database 150 may include data such as proportion of days covered (PDC), calculated as a ratio of the number of days in a period covered to the number of days in a given period for each member and corresponding medication.

In an example implementation, the dataset 151 described herein as accessed from the provider database 145 and/or member database 150 may include claims-based electronic data and/or prescription-based electronic data (also referred to herein as claims-based data and prescription-based data, respectively). For example, the dataset 151 may include claims-based electronic data including Current Procedural Terminology (CPT) codes and National Drug Code (NDC) numbers. In the example implementation, the dataset 151 may be absent (e.g., not include) measured values of biomarkers. In another example implementation, the dataset 151 may include some measured values of a biomarker, but the quantity of the measured values may be below a threshold value for determining (or accurately determining) the health status.

The server 135 may include a processor 160, a network interface 165, a database interface 170, and a memory 175. In some examples, components of the server 135 (e.g., processor 160, a network interface 165, a database interface 170, and a memory 175) may communicate via a system bus (e.g., any combination of control busses, address busses, and data busses) included in the server 135. Aspects of the processor 160, network interface 165, database interface 170, and memory 175 may support example functions of the server 135 as described herein. For example, the server 135 may transmit packets to (or receive packets from) one or more other devices (e.g., one or more communication devices 105, another server 135, the provider database 145, the provider database 150) via the communication network 140. In some aspects, via the network interface 165, the server 135 may transmit database queries to one or more databases (e.g., provider database 145, member database 150) of the system 100, receive responses associated with the database queries, or access data associated with the database queries.

In some aspects, via the network interface 165, the server 135 may transmit one or more electronic communications 155 described herein to one or more communication devices 105 of the system 100. The network interface 165 may include, for example, any combination of network interface cards (NICs), network ports, associated drivers, or the like. Communications between components (e.g., processor 160, network interface 165, database interface 170, and memory 175) of the server 135 and other devices (e.g., one or more communication devices 105, the provider database 145, the provider database 150, another server 135) connected to the communication network 140 may, for example, flow through the network interface 165.

The processors described herein (e.g., processor 110 of the communication device 105, processor 160 of the server 135) may correspond to one or many computer processing devices. For example, the processors may include a silicon chip, such as a Field Programmable Gate Array (FPGA), an Application-Specific Integrated Circuit (ASIC), any other type of Integrated Circuit (IC) chip, a collection of IC chips, or the like. In some aspects, the processors may include a microprocessor, a Central Processing Unit (CPU), a Graphics Processing Unit (GPU), or plurality of microprocessors configured to execute the instructions sets stored in a corresponding memory (e.g., memory 120 of the communication device 105, memory 175 of the server 135). For example, upon executing the instruction sets stored in memory 120, the processor 110 may enable or perform one or more functions of the communication device 105. In another example, upon executing the instruction sets stored in memory 175, the processor 160 may enable or perform one or more functions of the server 135.

The processors described herein (e.g., processor 110 of the communication device 105, processor 160 of the server 135) may utilize data stored in a corresponding memory (e.g., memory 120 of the communication device 105, memory 175 of the server 135) as a neural network. The neural network may include a machine learning architecture. In some aspects, the neural network may be or include one or more classifiers. In some other aspects, the neural network may be or include any machine learning network such as, for example, a deep learning network, a convolutional neural network, or the like. Some elements stored in memory 120 may be described as or referred to as instructions or instruction sets, and some functions of the communication device 105 may be implemented using machine learning techniques. In another example, some elements stored in memory 175 may be described as or referred to as instructions or instruction sets, and some functions of the server 135 may be implemented using machine learning techniques.

In some aspects, the processors (e.g., processor 110, processor 160) may support machine learning model(s) 184 which may be trained and/or updated based on data (e.g., training data 186) provided or accessed by any of the communication device 105, the server 135, the provider database 145, and the member database. The machine learning model(s) 184 may be built and updated by any of the engines described herein (e.g., prediction engine 183) based on the training data 186 (also referred to herein as training data and feedback). For example, the machine learning model(s) 184 may be trained with feature vectors of members (e.g., accessed from provider database 145 or member database 150) for which claims-based data (and/or prescription-based data) and recorded biomarker data (e.g., values of biomarkers) corresponded to recorded health statuses of the members.

In some aspects, the training data 186 may include multiple training sets. For example, the machine learning model(s) 184 may be trained with a first training set that includes feature vectors of members (e.g., accessed from provider database 145 or member database 150) for which a first set of claims-based data and/or a first set of prescription-based data resulted in a first set of biomarker data (e.g., biomarker values) associated with a relatively positive impact (e.g., positive clinical impact, progression to a positive medical diagnosis, prevention of a negative medical diagnosis, etc.).

In an example, the machine learning model(s) 184 may be trained with a second training set that includes feature vectors of members for which a second set of claims-based data and/or a second set of prescription-based data resulted in a second set of biomarker data associated with a relatively negative impact (e.g., negative clinical impact, progression to a negative medical diagnosis, etc.).

In another example, aspects of the present disclosure include training the machine learning model(s) 184 with a third training set that includes feature vectors of members (e.g., accessed from provider database 145 or member database 150) for which a diagnosed medical condition was correlated to measured biometric information of the members.

In some other examples, aspects of the present disclosure include creating a fourth training set based on data included in any of the first through third training sets.

In some aspects, training the machine learning model(s) 184 may be based on a target prediction accuracy of the machine learning model(s) 184. For example, training may include building and validating the machine learning model(s) 184 for generalized biomarker prediction, disease prediction, and treatment efficacy prediction. In some examples, training the machine learning model(s) 184 and prediction using the machine learning model(s) 184 may be implemented using GPU enabled edge nodes (e.g., at a communication device 105, at the server 135, etc.).

The machine learning model(s) 184 may be provided in any number of formats or forms. Example aspects of the machine learning model(s) 184, such as generating (e.g., building, training) and applying the machine learning model(s) 184, are described with reference to the figure descriptions herein.

Non-limiting examples of the machine learning model(s) 184 include Decision Trees, gradient-boosted decision tree approaches (GBMs), Support Vector Machines (SVMs), Nearest Neighbor, and/or Bayesian classifiers, and neural-network-based approaches.

In some aspects, the machine learning model(s) 184 may include ensemble classification models (also referred to herein as ensemble methods) such as gradient boosting machines (GBMs). Gradient boosting techniques may include, for example, the generation of decision trees one at a time within a model, where each new tree may support the correction of errors generated by a previously trained decision tree (e.g., forward learning). Gradient boosting techniques may support, for example, the construction of ranking models for information retrieval systems. A GBM may include decision tree-based ensemble algorithms that support building and optimizing models in a stage-wise manner.

According to example aspects of the present disclosure described herein, the machine learning model(s) 184 may include Gradient Boosting Decision Trees (GBDTs). Gradient boosting is a supervised learning technique that harnesses additive training and tree boosting to correct errors made by previous models, or regression trees.

The machine learning model(s) 184 may include extreme gradient boosting (CatBoost) models. CatBoost is an ensemble learning method based on GBDTs. In some cases, CatBoost methods may have improved performance compared to comparable random forest-based methods. CatBoost methods are easily tunable and scalable, offer a higher computational speed in comparison to other methods, and are designed to be highly integrable with other approaches including Shapley Additive Explanations (SHAP) values.

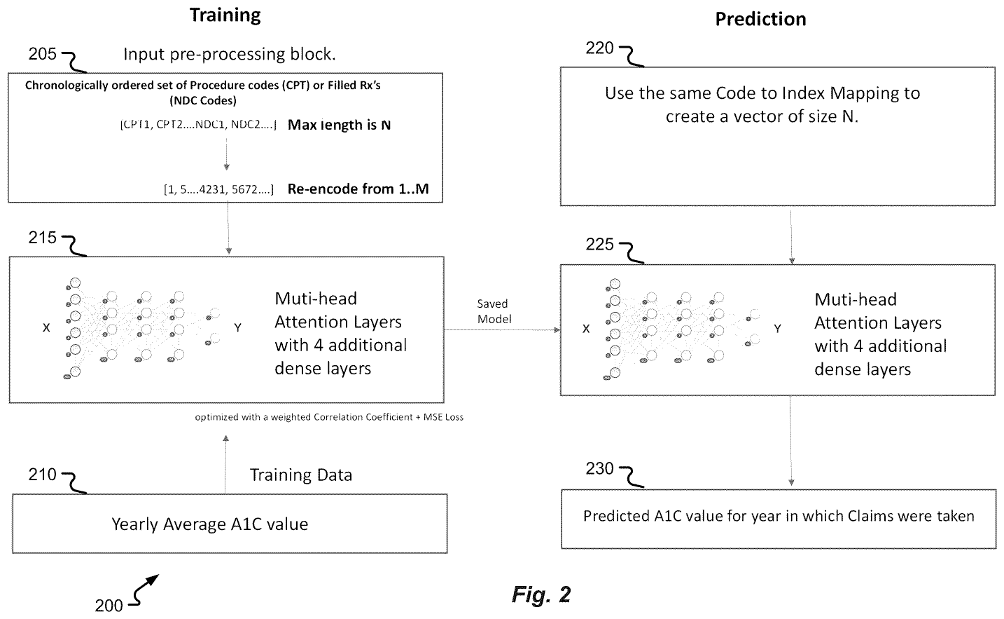

Examples implementations of training and prediction using neural networks and machine learning model(s) 184 of the system 100 are described herein with reference to FIGS. 2, 3, and 4A through 4C.

In some aspects, the machine learning model(s) 184 may include ensemble classification models (also referred to herein as ensemble methods) such as random forests. Random forest techniques may include independent training of each decision tree within a model, using a random sample of data. Random forest techniques may support, for example, medical diagnosis techniques described herein using weighting techniques with respect to different data sources.

Various example aspects of the machine learning model(s) 184, inputs to the machine learning model(s) 184, and the training data 186 with respect to the present disclosure are described here.

The memory described herein (e.g., memory 120, memory 175) may include any type of computer memory device or collection of computer memory devices. For example, a memory (e.g., memory 120, memory 175) may include a Random Access Memory (RAM), a Read Only a Memory (ROM), a flash memory, an Electronically-Erasable Programmable ROM (EEPROM), Dynamic RAM (DRAM), or any combination thereof.

The memory described herein (e.g., memory 120, memory 175) may be configured to store instruction sets, neural networks, and other data structures (e.g., depicted herein) in addition to temporarily storing data for a respective processor (e.g., processor 110, processor 160) to execute various types of routines or functions. For example, the memory 175 may be configured to store program instructions (instruction sets) that are executable by the processor 160 and provide functionality of any of the engines described herein.

The memory described herein (e.g., memory 120, memory 175) may also be configured to store data or information that is useable or capable of being called by the instructions stored in memory. Examples of data that may be stored in memory 175 for use by components thereof include machine learning model(s) 184 and/or training data 186 described herein.

Any of the engines described herein may include a single or multiple engines.

With reference to the server 135, the memory 175 may be configured to store instruction sets, neural networks, and other data structures (e.g., depicted herein) in addition to temporarily storing data for the processor 160 to execute various types of routines or functions. The illustrative data or instruction sets that may be stored in memory 175 may include, for example, database interface instructions 176, an electronic record filter 178 (also referred to herein as a feature vector filter), a feature embedding engine 179, a care gap management engine 182, and a reporting engine 188. In some examples, the reporting engine 188 may include data obfuscation capabilities 190 via which the reporting engine 188 may obfuscate, remove, redact, or otherwise hide personally identifiable information (PII) from an electronic communication 155 prior to transmitting the electronic communication 155 to another device (e.g., communication device 105).

In some examples, the database interface instructions 176, when executed by the processor 160, may enable the server 135 to send data to and receive data from the provider database 145, the member database 150, or both. For example, the database interface instructions 176, when executed by the processor 160, may enable the server 135 to generate database queries, provide one or more interfaces for system administrators to define database queries, transmit database queries to one or more databases (e.g., provider database 145, the member database 150), receive responses to database queries, access data associated with the database queries, and format responses received from the databases for processing by other components of the server 135.

The server 135 may use the electronic record filter 178 in connection with processing data received from the various databases (e.g., provider database 145, member database 150). For example, the electronic record filter 178 may be leveraged by the database interface instructions 176 to filter or reduce the number of electronic records (e.g., feature vectors) provided to any of the feature embedding engine 179, the care gap management engine 182, or the prediction engine 183. In an example, the database interface instructions 176 may receive a response to a database query that includes a set of feature vectors (e.g., a plurality of feature vectors associated with different members). In some aspects, any of the database interface instructions 176, the feature embedding engine 179, the care gap management engine 182, or the prediction engine 183 may be configured to utilize the electronic record filter 178 to reduce (or filter) the number of feature vectors received in response to the database query, for example, prior to processing data included in the feature vectors.

The feature embedding engine 179 may receive, as input, sequences of medical terms extracted from claim data (e.g., medical claims, pharmacy claims) for each member. In an example, the feature embedding engine 179 may process the input using neural word embedding algorithms such as Word2vec. In some examples, the feature embedding engine 179 may process the input using Transformer algorithms (e.g., algorithms associated with language models such as Bidirectional Encoder Representations from Transformers (BERT) or Generative Pre-trained Transformer (GPT) or graph convolutional transformer (GCT)) and respective attentional mechanisms. In some aspects, based on the processing, the feature embedding engine 179 may compute and output respective dimension weights for the medical terms. In some aspects, the dimension weights may include indications of the magnitude and direction of the association between a medical code and a dimension. In an example, the feature embedding engine 179 may compute an algebraic average of all the medical terms for each member over any combination of dimensions (e.g., over all dimensions). In some aspects, the algebraic average may be provided by the feature embedding engine 179 as additional feature vectors in a predictive model described herein (e.g., classifier).

The member grouping engine 180, when executed by the processor 160, may enable the server 135 to group data records of various members according to a common value(s) in one or more fields of such data records. For example, the member grouping engine 180 may group electronic records based on commonalities in parameters such as health conditions (e.g., diagnosis of diabetes, open gaps-in-care, closed gaps-in-care, suggested actions associated with closing a gap-in-care, impact associated with at least partially closing the gap-in-care, etc.), medical treatment histories, prescriptions, healthcare providers, locations (e.g., state, city, ZIP code, etc.), gender, age range, medical claims, pharmacy claims, lab results, medication adherence, demographic data, social determinants (also referred to herein as social indices), biomarkers, behavior data, engagement data, historical gap-in-care data, machine learning model-derived outputs, combinations thereof, and the like.

The reporting engine 188, when executed by the processor 160, may enable the server 135 to output one or more electronic communications 155 based on data generated by any of the feature embedding engine 179, the member grouping engine 180, the care gap management engine 182, or the prediction engine 183. The reporting engine 188 may be configured to generate electronic communications 155 in various electronic formats, printed formats, or combinations thereof. Some example formats of the electronic communications 155 may include HyperText Markup Language (HTML), electronic messages (e.g., email), documents for attachment to an electronic message, text messages (e.g., SMS, instant messaging, etc.), combinations thereof, or any other known electronic file format. Some other examples include sending, for example, via direct mail, a physical representation (e.g., a letter) of the electronic communication 155.

The reporting engine 188 may also be configured to hide, obfuscate, redact, or remove PII data from an electronic communication 155 prior to transmitting the electronic communication 155 to another device (e.g., a communication device 105, the server 135, etc.). The reporting engine 188 may also be configured to hide, obfuscate, redact, and/or remove PII data from an electronic data record prior to transmitting the electronic data record to another device (e.g., a communication device 105). In some aspects, a communication device 105 may also be configured to hide, obfuscate, redact, or remove PII data from direct mail (e.g., a letter) prior to generating a physical representation (e.g., a printout) of an electronic communication 155. In some examples, the data obfuscation may include aggregating electronic records to form aggregated member data that does not include any PII for a particular member or group of members. In some aspects, the aggregated member data generated by the data obfuscation may include summaries of data records for member groups, statistics for member groups, or the like.

Example illustrative aspects of the system 100 are described with reference to FIGS. 2, 3, and 4A through 4C.

FIG. 2 illustrates a block diagram 200 that supports aspects of the present disclosure. The block diagram 200 is described with reference to and may be implemented by aspects of the system 100 of FIG. 1.

Referring to FIG. 2, blocks 205 through 215 support aspects of training the machine learning model(s) 184 of FIG. 1. Blocks 220 through 235 support aspects of predicting biomarker values using the machine learning model(s) 184.

Block 205 may be an input pre-processing block. Block 205 may include preprocessing a dataset including a series of prescription codes and CPT codes. In an example of the preprocessing, the system 100 may order the prescription codes and CPT codes based on a set of criteria. In an example, the system 100 may chronologically order the prescription codes and CPT codes with respect to time.

Block 210 may include a training dataset. In an example, the training dataset may include measured values for a target biomarker(s) (e.g., A1C) over a temporal period (e.g., one year). The datasets included in association with block 205 and block 210 may be examples of aspects of the training data 186 of FIG. 1.

Block 215 may be an example of a neural network according to aspects of the present disclosure. Block 215 may include multiple deep learning layers. The deep learning layers may include multi-head attention layers and dense layers. The attention layers may enhance parts of the datasets provided from block 205 and block 210, while diminishing other parts, which may enable the neural network to focus on the enhanced parts of the datasets. The dense layers are layers that are deeply connected with respective preceding layers, such that for each dense layer, the neurons of the dense layer are connected to every neuron of the preceding layer.

In an example implementation, using the datasets of block 205 and block 210, the system 100 may train the neural network of block 215 using a population for which measured values of a biomarker (e.g., measured A1C values, measured cholesterol levels, measured blood pressure values, etc.) are available. For example, the system 100 may provide a trained model (e.g., a machine learning model(s) 184 of FIG. 1). In an example of training the neural network of block 215, the system 100 may encode the data from the datasets of block 205 and block 210 such that, for example, the most common data among the training datasets is assigned a ‘1’, the next common data in the training dataset is assigned a ‘2’, and so on.

According to example aspects of the present disclosure, using the trained neural network and trained model, the system 100 is able to predict values of a biomarker (e.g., predict A1C values, predict cholesterol levels, predict blood pressure values, etc.) for other populations for which no measured values of the biomarker are available.

For example, block 220 may include a dataset (e.g., claims-based data, prescription-based data, etc.) of a population for which no corresponding measured values of a biomarker are available. At block 225, the system 100 may process the dataset of block 220 using the trained neural network. The trained neural network may output predicted values 230 of the biomarker, for example, for a temporal duration (e.g., a one year period) corresponding to the dataset.

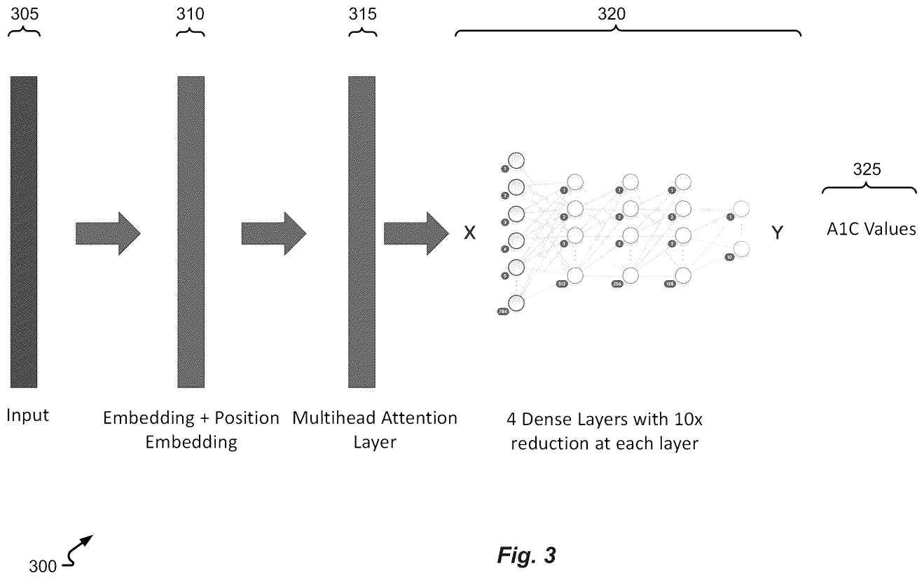

FIG. 3 illustrates an example 300 of a neural network architecture in accordance with aspects of the present disclosure. The example 300 is described with reference to and may be implemented by aspects of the system 100 of FIG. 1.

The neural network may include an input layer 305, an embedding and position embedding layer(s) 310, a multi-head attention layer(s) 315, and a set of dense layers 320 (e.g., 4 dense layers with 10× reduction at each dense layer). The neural network may process a dataset (e.g., claims-based data, prescription-based data, etc.) of a population for which no corresponding measured values of a biomarker (e.g., A1C) are available. Based on the processing, the neural network may provide an output 325 including predicted values of the biomarker for a temporal duration corresponding to the dataset.

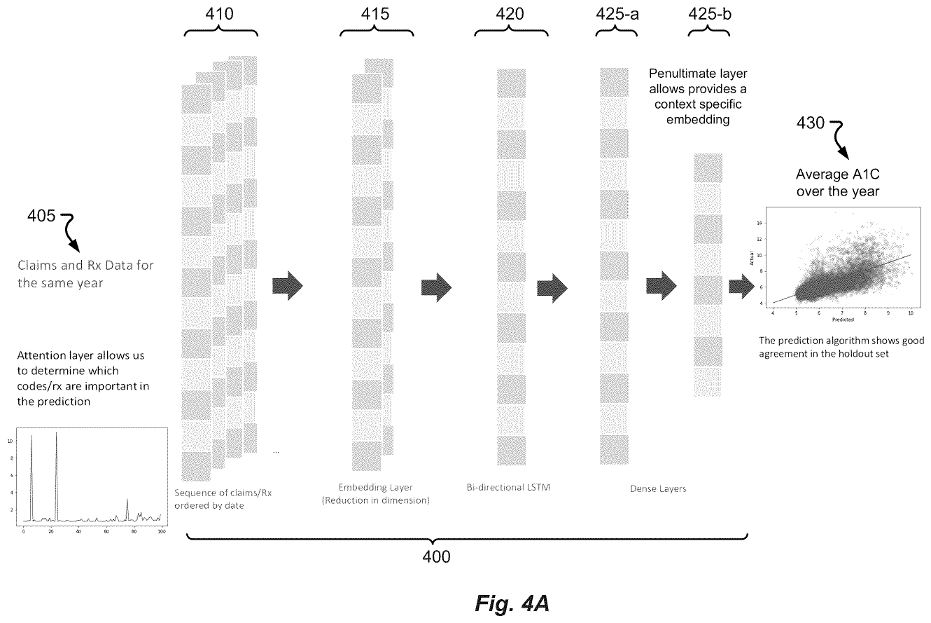

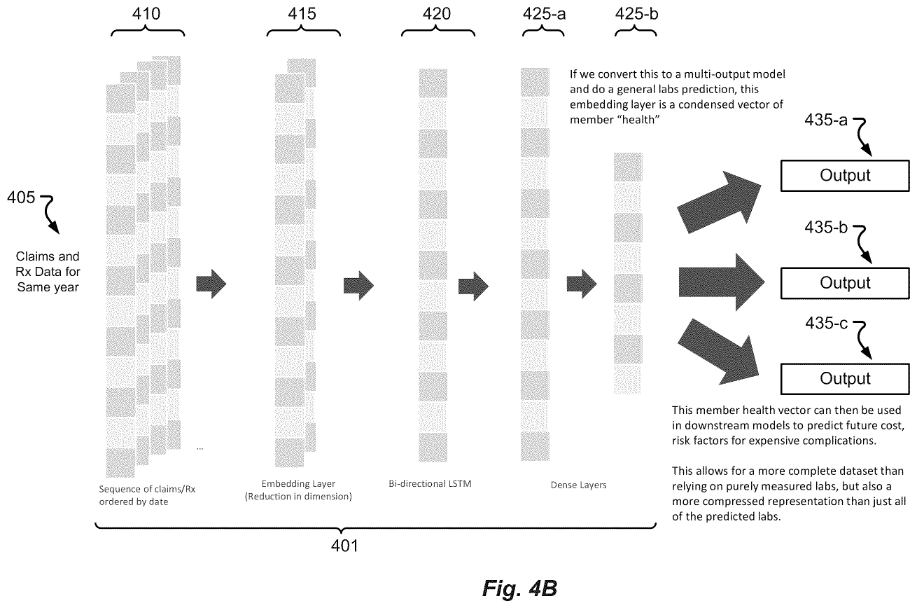

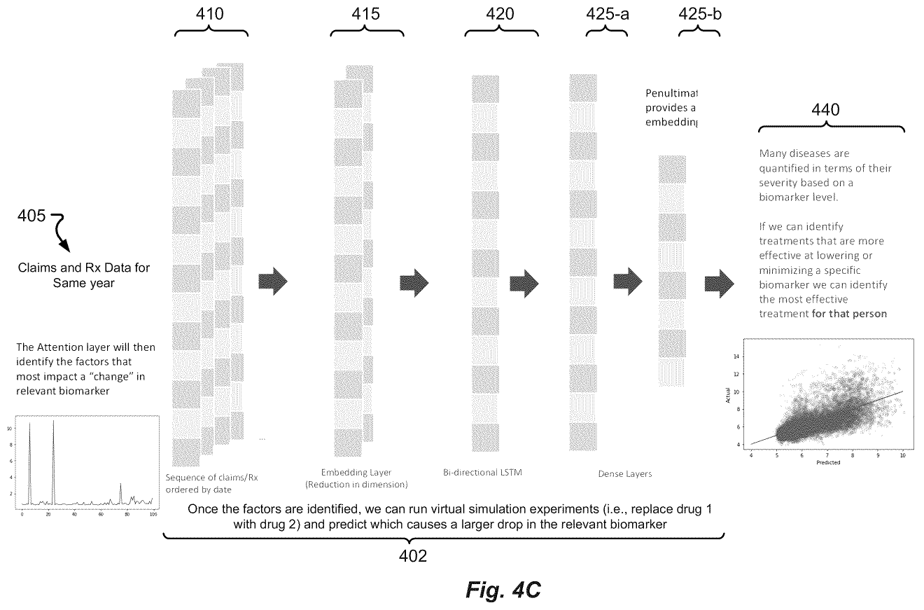

FIGS. 4A through 4C illustrate example implementations 400 through 402 of a neural network architecture in accordance with aspects of the present disclosure. The example implementations 400 through 402 may be implemented by aspects of the system 100 of FIG. 1

Referring to the example implementation 400 of FIG. 4A, the system 100 may receive input data 405 (e.g., claims-based data and/or prescription-based data for the same temporal duration, for example, a one year period).

The system 100 may preprocess the input data 405 and provide data 410. Data 410 may include the claims-based data and/or prescription-based data, ordered based on respective dates associated with the claims-based data and/or prescription-based data.

The system 100 may process the data 410 using embedding layer(s) 415, bi-directional long short-term memory (LSTM) 420, and dense layers 425 (e.g., dense layer 425-a and dense layer 425-b). In an example, dense layer 425-b may be fully-connected hidden layer (e.g., a penultimate layer).

In the example implementation 400, the system 100 may provide an output 430 including an average value of a biomarker (e.g., A1C) with respect to the same temporal duration as the input data 405. In some aspects, the system 100 may verify the output 430 by comparing the predicted values of the biomarker to measured values of the biomarker.

Additionally, or alternatively, referring to the example implementation 401 of FIG. 4B, the system 100 may support a multi-output mode. For example, the system 100 may provide outputs 435 (e.g., output 435-a, output 43-b, output 435-c) for the same temporal duration as the input data 405. In some examples, the outputs 435 may be member health vectors (e.g., predicted health statuses) of a member for the same temporal duration as the input data 405.

Additionally, or alternatively, referring to the example implementation 402 of FIG. 4C, the system 100 may support prediction and identification of treatment plans that may be effective at impacting (e.g., reducing, minimizing, increasing, maintaining, etc.) the value of a target biomarker, and thereby, be effective at treating a medical condition associated with the target biomarker. Examples of the treatment plans may include a medical procedure, an action of the member (e.g., diet, exercise, etc.), and a drug regimen, but are not limited thereto.

For example, the system 100 may provide an output 440 for the same temporal duration as the input data 405. In some examples, the output 440 may include candidate treatment plans, predicted biomarker values associated with implementing the candidate treatment plans, and measured biomarker values associated with implementing the candidate treatment plans.

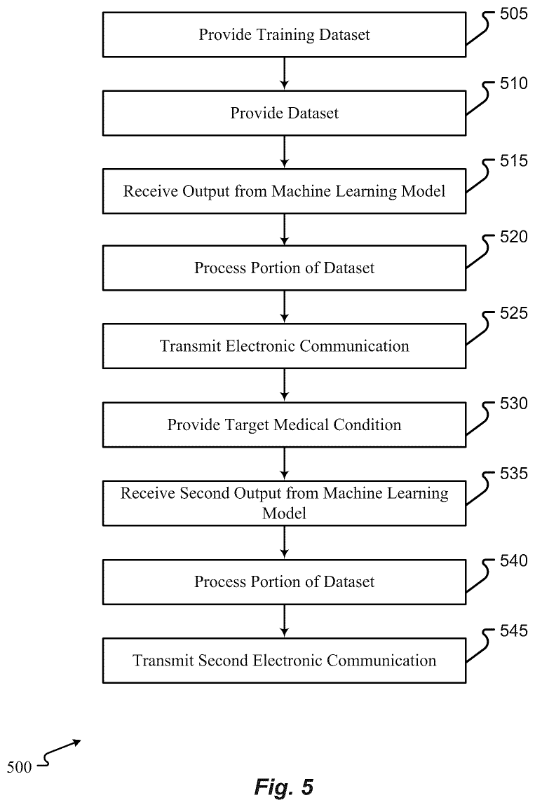

FIG. 5 illustrates an example of a process flow 500 that supports machine learning for predicting biomarkers in accordance with aspects of the present disclosure. In some examples, process flow 500 may implement aspects of a communication device 105 or a server 135 described with reference to FIGS. 1, 2, 3, and 4A through 4C.

In the following description of the process flow 500, the operations may be performed in a different order than the order shown, or the operations may be performed in different orders or at different times. Certain operations may also be left out of the process flow 500, or other operations may be added to the process flow 500.

It is to be understood that while a device 105 is described as performing a number of the operations of process flow 500, any device (e.g., another device 105 in communication with the device 105, a server 135, etc.) may perform the operations shown.

At 505, the process flow 500 may include providing a training dataset to a machine learning model (e.g., machine learning model(s) 184 of FIG. 1).

In an example, the training dataset includes claims-based electronic data of a set of reference individuals. In another example, the training dataset includes prescription-based electronic data of the set of reference individuals.

In some examples, the training dataset includes measured biometric data of the set of reference individuals, wherein the measured biometric data includes reference measured values of the biomarker.

In another example, the training dataset includes diagnosed medical conditions associated with the set of reference individuals.

At 510, the process flow 500 may include providing a dataset to a machine learning model.

In some aspects, the dataset includes claims-based electronic data. In some aspects, the claims-based electronic data is associated with a group of individuals. In some aspects, the claims-based electronic data includes Current Procedural Terminology (CPT) codes and National Drug Code (NDC) numbers (also referred to herein as CPT identifiers and NDC identifiers).

In some aspects, the dataset includes prescription-based electronic data.

At 515, the process flow 500 may include receiving an output from the machine learning model in response to the machine learning model processing at least a portion of the dataset, wherein the output includes a predicted value of the biomarker.

In some example aspects, the biomarker is associated with individuals in the group of individuals. In some aspects, the biomarker includes hemoglobin A1C. Additionally, or alternatively, the biomarker may include cholesterol level or blood pressure level.

In some aspects, the output includes a predicted health status of the at least one individual corresponding to the predicted value of the biomarker. In some examples, the predicted health status includes at least one of: a medical condition of the at least one individual; a predicted risk of the at least one individual with respect to developing the medical condition; and a predicted severity of the medical condition.

In some example aspects, the input includes one or more candidate intervention actions. In some cases, the output includes a predicted effect of each of the one or more candidate interventions. In some examples, the predicted effect includes: a second predicted value of the biomarker; and a temporal value associated with achieving the second predicted value.

In some example aspects, the input includes one or more candidate treatment plans. In some aspects, the one or more candidate treatment plans include at least one of: a medical procedure; an action of the at least one individual; and a drug regimen. In some example aspects, the output includes a predicted effect of each of the one or more candidate treatment plans. In an example, the predicted effect includes: a second predicted value of the biomarker; and a temporal value associated with achieving the second predicted value.

In some example aspects, the output includes respective rankings corresponding to implementing a candidate intervention action; and implementing a candidate treatment plan.

In some aspects, the output includes a range of values of the biomarker.

In some aspects, the output includes a predicted value of one or more additional biomarkers associated with at least one individual.

At 520, the process flow 500 may include processing the portion of the dataset for identifying information associated with the at least one individual in response to determining the predicted value of the biomarker satisfies one or more criteria.

At 525, the process flow 500 may include transmitting, via a communication network to one or more communication devices, an electronic communication including information associated with the predicted value of the biomarker.

In some aspects, the electronic communication includes at least a portion of the output from the machine learning model.

In some aspects, the one or more communication devices include a communication device of the at least one individual, a communication device of a care provider of the at least one individual, or both.

At 530, the process flow 500 may include providing a target medical condition to the machine learning model.

At 535, the process flow 500 may include receiving a second output from the machine learning model in response to providing the target medical condition.

In an example, the second output includes: one or more biomarkers indicative of the target medical condition; and a predicted value of the one or more biomarkers.

In some aspects, the second output includes one or more claims-based identifiers indicative of the target medical condition.

In some other aspects, the second output includes one or more prescription-based identifiers indicative of the target medical condition.

At 540, the process flow 500 may include processing the portion of the dataset for the identifying information associated with the at least one individual in response to determining the predicted value of the one or more biomarkers satisfies one or more second criteria.

At 545, the process flow 500 may include transmitting, via the communication network to the one or more communication devices, a second electronic communication including information associated with the predicted value of the one or more biomarkers.

A number of implementations have been described. Nevertheless, it will be understood that additional modifications may be made without departing from the scope of the inventive concepts described herein, and, accordingly, other examples are within the scope of the following claims.

The exemplary systems and methods of this disclosure have been described in relation to examples of a communication device 105 and a server 135. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

Furthermore, while the examples illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed examples, configuration, and aspects.

A number of variations and modifications of the disclosure can be used. It would be possible to provide for some features of the disclosure without providing others.

In yet another example, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another examples, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another example, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the examples with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various examples, configurations, and aspects, includes components, methods, processes, systems and/or apparatus substantially as depicted and described herein, including various examples, subcombinations, and subsets thereof. Those of skill in the art will understand how to make and use the systems and methods disclosed herein after understanding the present disclosure. The present disclosure, in various examples, configurations, and aspects, includes providing devices and processes in the absence of items not depicted and/or described herein or in various examples, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more examples, configurations, or aspects for the purpose of streamlining the disclosure. The features of the examples, configurations, or aspects of the disclosure may be combined in alternate examples, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed example, configuration, or aspect. Thus, the following claims are hereby incorporated into this Detailed Description, with each claim standing on its own as a separate preferred example of the disclosure.

Moreover, though the description of the disclosure has included description of one or more examples, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative examples, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an example that is entirely hardware, an example that is entirely software (including firmware, resident software, micro-code, etc.) or an example combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A method comprising:
providing a dataset to a machine learning model, wherein the dataset comprises claims-based electronic data;
receiving an output from the machine learning model in response to the machine learning model processing at least a portion of the dataset, wherein the output comprises a predicted value of a biomarker;
processing the portion of the dataset for identifying information associated with at least one individual in response to determining the predicted value of the biomarker satisfies one or more criteria; and
transmitting, via a communication network to one or more communication devices, an electronic communication comprising information associated with the predicted value of the biomarker.

2. The method of claim 1, wherein:
the claims-based electronic data is associated with a group of individuals; and
the biomarker is associated with individuals in the group of individuals.

3. The method of claim 1, wherein the dataset comprises prescription-based electronic data.

4. The method of claim 1, wherein the biomarker comprises hemoglobin A1C.

5. The method of claim 1, wherein the dataset does not include measured values of the biomarker.

6. The method of claim 1, wherein the output comprises:
a predicted health status of the at least one individual corresponding to the predicted value of the biomarker.

7. The method of claim 6, wherein the predicted health status comprises at least one of:
a medical condition of the at least one individual;
a predicted risk of the at least one individual with respect to developing the medical condition; and
a predicted severity of the medical condition.

8. The method of claim 1, further comprising:
providing a training dataset to the machine learning model, wherein the training dataset comprises:
claims-based electronic data of a set of reference individuals;
prescription-based electronic data of the set of reference individuals;
measured biometric data of the set of reference individuals, wherein the measured biometric data comprises reference measured values of the biomarker; and
diagnosed medical conditions associated with the set of reference individuals.

9. The method of claim 1, wherein:
the input comprises one or more candidate intervention actions; and
the output comprises a predicted effect of each of the one or more candidate interventions, wherein the predicted effect comprises:
a second predicted value of the biomarker; and
a temporal value associated with achieving the second predicted value.

10. The method of claim 1, wherein the input comprises:
one or more candidate treatment plans; and
the output comprises a predicted effect of each of the one or more candidate treatment plans, wherein the predicted effect comprises:
a second predicted value of the biomarker; and
a temporal value associated with achieving the second predicted value.

11. The method of claim 10, wherein the one or more candidate treatment plans comprise at least one of:
a medical procedure;
an action of the at least one individual; and
a drug regimen.

12. The method of claim 1, wherein the output comprises respective rankings corresponding to:
implementing a candidate intervention action; and
implementing a candidate treatment plan.

13. The method of claim 1, further comprising:
providing a target medical condition to the machine learning model;
receiving a second output from the machine learning model in response to providing the target medical condition, wherein the second output comprises:
one or more biomarkers indicative of the target medical condition; and
a predicted value of the one or more biomarkers;
processing the portion of the dataset for the identifying information associated with the at least one individual in response to determining the predicted value of the one or more biomarkers satisfies one or more second criteria; and
transmitting, via the communication network to the one or more communication devices, a second electronic communication comprising information associated with the predicted value of the one or more biomarkers, wherein the second output comprises at least one of:
one or more claims-based identifiers indicative of the target medical condition; and
one or more prescription-based identifiers indicative of the target medical condition.

14. The method of claim 1, wherein the claims-based electronic data comprises Current Procedural Terminology (CPT) codes and National Drug Code (NDC) numbers.

15. The method of claim 1, wherein the output comprises a range of values of the biomarker.

16. The method of claim 1, wherein the output comprises a predicted value of one or more additional biomarkers associated with the at least one individual.

17. The method of claim 1, wherein the electronic communication comprises at least a portion of the output from the machine learning model.

18. The method of claim 1, wherein the one or more communication devices comprise a communication device of the at least one individual, a communication device of a care provider of the at least one individual, or both.

19. A system comprising:
a communications interface;
a processor coupled with the communications interface; and
a memory coupled with the processor, wherein the memory stores data that, when executed by the processor, enables the processor to:
receive an output from a machine learning model in response to the machine learning model processing at least a portion of a dataset, wherein the dataset comprises claims-based electronic data, and wherein the output comprises a predicted value of a biomarker;
process the portion of the dataset for identifying information associated with at least one individual in response to determining the predicted value of the biomarker satisfies one or more criteria; and
transmit, via the communications interface and a communication network to one or more communication devices, an electronic communication comprising information associated with the predicted value of the biomarker.

20. A non-transitory computer-readable medium comprising instructions stored therein that, when executed by a processor, cause the processor to:
receive an output from a machine learning model in response to the machine learning model processing at least a portion of a dataset, wherein the dataset comprises claims-based electronic data, and wherein the output comprises a predicted value of a biomarker;
process the portion of the dataset for identifying information associated with at least one individual in response to determining the predicted value of the biomarker satisfies one or more criteria; and
transmit, via a communication network to one or more communication devices, an electronic communication comprising information associated with the predicted value of the biomarker.
